(19) United States
(12) Patent Application Publication
(10) Pub. No.: US 2013/0005591 A1
(43) Pub. Date: Jan. 3, 2013

(54) METHOD FOR PARALLEL AMPLIFICATION OF NUCLEIC ACIDS

(75) Inventor: Wei-Wen Cai, Allen, TX (US)

(73) Assignee: HALGEN CORPORATION, Plano (US)

(21) Appl. No.: 13/349,780

(22) Filed: Jan. 13, 2012

Related U.S. Application Data
(60) Provisional application No. 61/432,351, filed on Jan. 13, 2011.

(51) Int. Cl. C40B 50/06; C40B 30/04; C12P 19/34

(52) U.S. Cl. 506/9; 506/26; 435/91.2

(57) ABSTRACT

A new method that enables parallel amplification of nucleic sequences from a complex source is disclosed. The amplification is compartmentized into microdroplets by porous-walled hollow glass microspheres and subjected to PCR thermal cycling or isothermal amplification. The rigid wall of the glass microspheres allows very simple and conventional manipulation of the amplification products for downstream application such as sequencing or detection of copy number of specific sequences in a complex sample.

### FIELD OF THE INVENTION

The present invention is in the technical field of genetic analysis. More specifically, the present invention relates to methods for amplifying nucleic acid templates in a massively parallel manner from individually sequestered molecules to a high copy number amenable for sequencing and other applications such as detection of the copy number of specific sequences in a complex source.

### BACKGROUND OF THE INVENTION

Genome sequencing has revolutionized modern molecular biology. Conventional high-throughput genome sequencing strategy relies on a concerted effort of a team of technicians running a series of automatic or semiautomatic sequencing machines. A substantial work load in this sequencing strategy is to prepare nucleic acid samples carrying short individual fragments from a genome. The sample preparation process involves cloning individual fragments in a proper cloning vector and the clones are amplified for nucleic acid preparation for further sequencing reaction. While this strategy is immensely successful in obtaining high quality sequences for many genomes today, application of this strategy to a larger scale is not feasible because of the high cost and the limited throughput in nucleic acid sample preparation.

New sequencing strategies developed in recent years have obviated this lengthy and costly sample preparation process. Sequencing systems based on single molecule detection can even directly obtain sequence information from single nucleic acid molecules without amplification (Harris T D et al. Science. 2008 Apr. 4; 320(5872):106-9; Orlando L, et al. Genome Res. 2011 Oct.; 21(10):1705-19. Epub 2011 Jul. 29; Steinmann K E et al. Methods Mol. Biol. 2011; 733:3-24). However, single molecule sequencing methods are prone to errors and currently dominant next-generation sequencing systems still requires clonal amplification of individual nucleic acid fragments for better detection sensitivity and higher sequencing accuracy.

One method for parallel amplification of a large number of nucleic acid fragments is through encapsulating a plurality of DNA samples individually in a microcapsule of an emulsion (U.S. Pat. No. 6,489,103; Griffiths and Tawfik, EMBO, 22, pp. 24-35 (2003); Ghadessy et al., Proc. Natl. Acad. Sci. USA 98, pp. 4552-4557 (2001); Williams R et al. Nat. Methods. 3(7):545-50 (2006)), performing amplification of the plurality of encapsulated nucleic acid samples simultaneously, and releasing the amplified plurality of DNA from the microcapsules for subsequent reactions (U.S. Pat. Nos. 7,842,457; 7,947,477; 8,012,690). To facilitate the processing of clonally amplified DNA templates, in practice, single copies of the nucleic acid template species are hybridized to DNA capture beads, suspended in complete amplification solution and emulsified into microreactors (typically 100 to 200 microns in diameter), after which amplification (e.g., PCR) is used to clonally increase copy number of the initial template species to more than 1,000,000 copies of a single nucleic acid sequence (U.S. Pat. No. 7,842,457). While this sample preparation method has been incorporated in a commercial sequencing system, it has many limitations. A major disadvantage is that because beads are encapsulated in emulsified droplets of highly variable sizes the amplification level for different beads is highly variable. Another major limitation is that the emulsification process is very difficult to control, making the method very complicated and less reliable. Thus, it is clear that there exists a need for robust methods for preparation of whole genome libraries comprising of clonally amplified fragments without the time consuming sample preparation process and expensive, error-prone cloning processes.

DNA sequencing to detect mutations in disease genes has been a powerful diagnostic tool and formed the foundation for personalized medicine. In such application, gene fragments are usually amplified in individual PCR reaction and then processed individually for sequencing reaction and sequencing analysis by electrophoresis. Methods for parallel amplification of gene fragments by multiplex PCR (M. Edwards et al., PCR Methods and Applications, 3:565-575 (1994)), followed by hybridization onto microarray to detect sequence variations or mutation have been described (For example, see U.S. Pat. No. 5,882,856). However, designing a large number of compatible PCR primers for multiplexed PCR reaction have been proven to be very difficult and even not possible in most cases. And the expense for procuring a large number of primers becomes inhibitory. Thus, the development of less expensive but highly parallel amplification of a large number of targets is highly desirable for genetic diagnostic purpose.

Rapid and accurate detection of gene copy numbers is also an increasingly important practice in clinical diagnostics as well as research. Current methods of gene copy number detection include quantitative polymerase reaction and hybridization based procedures. While both methods are widely used they all suffer from low accuracy in resolving less variable copy number. Array based comparative hybridization has better resolution in copy number detection but requires higher amount of input DNA. Quantitative polymerase chain reaction has superior detection sensitivity but poor resolution in copy number detection. For example, quantitative polymerase chain reaction is not very reliable for detection of sequence duplications in a genome such as the detection of trisomy chromosome 21 in Down syndrome patients.

### SUMMARY OF THE INVENTION

The present invention is a novel method for parallel amplification of nucleic acids without emulsion and the encapsulated solid microbeads. The new method has the advantages of high amplification consistency and efficiency, and cost effective. This method has the potential to be formatted to perform multiplexed PCR on complex genomic DNA without the concern of incompatibility of PCR primers. The present invention, in essence, is single tube massively parallel PCR reactions effected by porous wall hollow glass microspheres, which serve as the micro reaction vessels isolated by a water immiscible phase. The stiff wall of the hollow glass microspheres offers the advantage of easy manipulation before and after PCR reaction, avoiding the problem of emulsion stability in bead emulsion PCR (U.S. Pat. No. 8,012,690).

### BRIEF DESCRIPTION OF THE DRAWINGS

FIG. 1 Illustrates the major steps in applying porous-walled hollow glass microspheres for clonal amplification of nucleic acid fragment using polymerase chain reaction.

FIG. 2. Illustrates the results of loading DNA fragments of different sizes to porous-walled hollow glass microspheres using two methods of loading. Lane 1 and 3, 100 bp size ladder directly loaded to gel wells; lane 2, porous-walled hollow glass microspheres (PW-HGMs) loaded by soaking in 100 bp size ladder solution and the fragments enter the PW-HGMs by passive diffusion through the porous walls; lane 4, PW-HGMs loaded with 100 bp size ladder under the influence of an electric field which actively move the fragments through the porous walls.

FIG. 3. Illustrate results of PW-HGM based PCR. Lane 1-5, Taq polymerase with different preparation of PW-HGMs. Lane 6, truncated Taq, PW-HGMs same as used in lane 3. Lane 7, (+) control without PW-HGMs, Lane 8, (−) control. Note the retarding effect of porous wall on the mobility of DNA.

FIG. 4. Illustrate the detection of cross PCR contamination by YOYO-3 staining. A, phase contrast image of amplified products of mixed PW-HGMs with DNA templates and without. B, fluorescent image of the same field. Some PW-HGMs showed no fluorescence signals indicating no cross contamination.

### DETAILED DESCRIPTION OF THE INVENTION

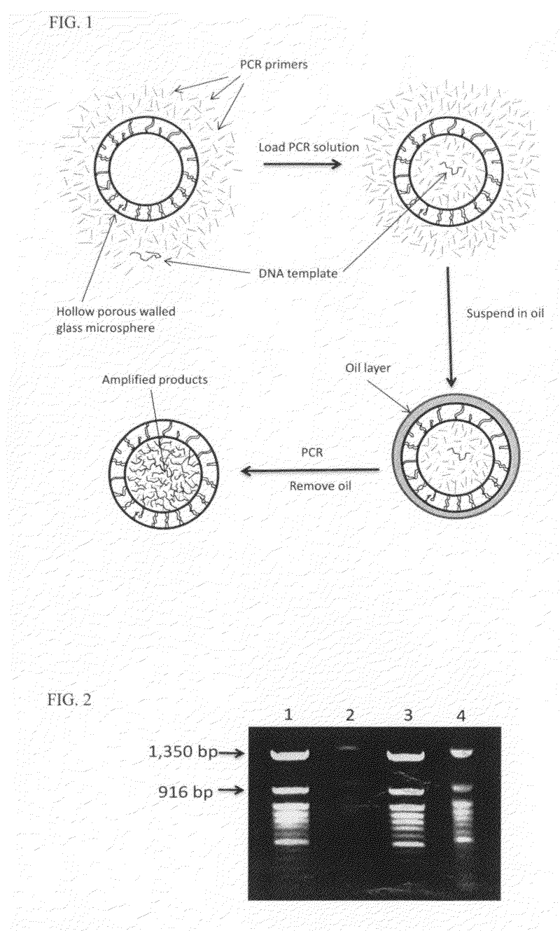

The process of conducting parallel amplification of nucleic acid fragments from a complex source is disclosed in the present invention. The amplification method consists of four major steps:
1. Loading a complete amplification reaction solution to fill up the void of porous-walled hollow glass microspheres (PW-HGMs).
2. The amplification solution loaded PW-HGMs are rinsed in an amplification buffer to remove excess amplification solution outside the PW-HGMs.
3. The PW-HGMs are suspended in a water immiscible phase such as mineral oil and subject the mixture to thermal cycling to start the amplification process.
4. The PW-HGMs are recovered after amplification by removing the water immiscible phase and rinsing with a detergent solution.
   An example of the amplification process is depicted in FIG. 1.

The present invention employs porous walled hollow glass microspheres (PW-HGMs) as the microreaction vessels for parallel amplification of nucleic acid templates. The process of preparing PW-HGMs has been previously described (U.S. Pat. No. 7,666,807; Li S, et al., Nanomedicine. 6(1):127-36 (2010)). Briefly, PW-HGMs are prepared by first annealing hollow glass microspheres at a critical softening temperature to induce phase separation in the wall of the glass microspheres. The wall is then made porous by leaching out the acid solvable borate-rich phase in an acid solution. The size of the pores in the wall can be controlled by the extent of annealing and/or the time of leaching. In the present invention, PW-HGMs 2-300 microns in diameter with a density ranging from 0.1 to 0.8 g/cc can be used and the wall pore size ranges from 5 to 900 nm.

In order to use the PW-HGMs as microreactors for amplification reaction, all the components in the amplification reaction must be able to enter the interior of the PW-HGMs. In one embodiment, this is done by simply incubating dry PW-HGMs with the amplification solution containing all necessary components which include primers, template nucleic acid molecules, DNA polymerase, and nucleotide triphosphates (dNTPs). The nucleic acid templates to be amplified are a population of DNA, such a genomic DNA fragment library, a cDNA library or a fragment library derived from other sources such bacterial genomes, viral genomes, plant genomes or subclones of complex genomes such bacterial artificial chromosome. In one embodiment, each template DNA molecule in the population has a first common adaptor sequence at one end and a second common adaptor sequence at the other end. The size of the DNA templates ranges from 100 to 2,000 bp.

In a preferred embodiment, the method for amplification is polymerase chain reaction. But in loading the amplification components to the void of the PW-HGMs, preferably, one essential component (usually dNTPs or Mg²⁺) are left out to prevent any reaction between the PCR primers during incubation at 4-10° C. The incubation time can be 2-20 hours depending on the size of the average DNA templates and the porosity of the porous wall. After all the components except dNTPs or Mg²⁺ are loaded, the PW-HGMs are transferred to a solution containing dNTPs or Mg²⁺ to complete the amplification solution. In another embodiment, the PW-HGMs are incubated with “Hot Start” PCR reaction solution which contains all the amplification components including primers, templates, buffer, dNTPs and DNA polymerase in a dormant state at room temperature. The major advantage of hot start PCR is that the polymerase only becomes active when the thermal cycling starts. This will reduce the risk of formation of primer-dimers, which is deleterious to amplification from a single template molecule (Chou et al., Nucleic Acids Research 20(7):1717-1723 (1992)).

There are several formats of “Hot Start” PCR which are all designed to prevent the non-specific PCR products due to primer-primer interaction at room temperature in the presence of DNA polymerase and dNTPs. However, in this invention, two formats of “Hot Start” PCR are preferred to keep the reaction mix dormant prior to thermal cycling. One format uses a form of DNA polymerase that is not active at room temperature but resumes full activity after treating at 94° C. for a few minutes (U.S. Pat. No. 5,773,258; Sharkey et al., Biotechnology (NY) 12:506-509 (1994); Kellogg et al., Biotechniques 16:1134-1137 (1994); U.S. Pat. No. 5,338,671; U.S. Pat. No. 5,985,619). In another “Hot Start” PCR format, normal Taq DNA polymerase is used but the nucleotides (dNTPs) are modified such that the 3′ hydroxyl groups are blocked with a thermal labile protecting group. The protected dNTPs will only become active after heating at 94° C. for a few minutes. These 3′ blocked dNTPs are now available from TriLink Technologies.

The porous wall of hollow glass microspheres, although permeable to small molecules such as dNTPs, may pose a barrier to large molecules like large template nucleic acids. This can result in an amplification bias towards smaller fragments. In a preferred embodiment, when the template is smaller than 500 bp, simple incubation is sufficient to allow the template to diffuse through the porous wall and enter the internal void of the microspheres. In an alternative embodiment, electric field can be used to actively move both small and large DNA templates across the porous wall.

The rigidity of the PW-HGMs offers great convenience in manipulation. PW-HGMs can be easily rinsed of the excess external solution without significant loss of internal content. This is usually done by briefly rinsing the amplification solution loaded PW-HGMs with amplification buffer. When such PW-HGMs are suspended in a water immiscible phase such as mineral oil all the PW-HGMs will be sealed by a thin layer of oil on the outer surface of the PW-HGMs (FIG. 1) and thus they will serve as independent microreaction for amplification. If each PW-HGM contains no more than 1 template molecule, amplification will be clonal—that is all copies of the amplified products are derived from a single template molecule in a single PW-HGM. This clonal amplification capability is particularly desirable for DNA sequencing and accurate detection of copy number of specific sequence in a complex nucleic acid samples such as a genome or transcriptom.

In preparing clonally amplified fragment libraries for DNA sequencing application, one primer may be attached to the PW-HGMs and a second primer is mixed in the amplification solution. After amplification, the amplified product will be attached to the PW-HGMs. As the surface area for primer binding includes the external, internal, and the surface area inside the channel in the porous wall, the available surface area of a porous wall glass microsphere is much greater than that of a solid bead of the same size. It has been estimated that the surface area of a PW-HGM is about 30 times that of a solid bead of the same size (U.S. Pat. No. 7,666,807). Therefore, PW-HGMs will offer much better sensitivity for fragment amplification than solid beads which are currently used in some commercial sequencing systems.

There are many ways to attach a first primer to the PW-HGMs. One common method is through the biotin-avidin linkage. The PW-HGMs is treated with amine containing silane such as 3-aminopropyltrimethoxysilane to confer amine groups on the surface of the PW-HGMs. A biotin compound with amine specific reactivity is then used to confer biotin moieties to the PW-HGMs surface. By incubating such biotin coated PW-HGMs in a solution containing avidin or strepavidin, the biotin molecules on the surface will be bound by avidin or strepavidin, which on contact with a primer with a biotin at the 5′ end of the primer will form a bond that is sufficiently stable for PCR reaction and post PCR processing. An alternative strategy for coupling a primer to the surface is to through the condensation reaction between amine group and carboxylate group in the presence of a water-soluble carbodiimide. In such case, the surface can be modified with a carboxylate silane compound and then coupled to the primer with an amine group at the 5′ end. Incubation of the amine labeled primer with the carboxylate coated PW-HGMs in the presence of condensation mediating agent 1-ethyl-3-(3-dimethylaminopropyl)carbodiimide (EDC) will create a stable covalent amide bond thus linking the primer to the PW-HGMs. Alternatively, primers with a 5′ phosphate group can be coupled to PW-HGMs coated with amine groups.

In a different embodiment, none of the primers is attached to PW-HGMs during PCR reaction. The amplified product is attached to the PW-HGMs after the amplification. In this format, one primer is biotin labeled at the 5′ end and the PW-HGMs are also coated with biotin as described above. After amplification, avidin or strepavidin is added to form links between the amplified products and the PW-HGMs. This embodiment is preferred when large fragments need to be clonally amplified. Primers in free solution always have high efficiency than in a bound state. The larger products will be preferentially retained inside the PW-HGMs in the washing steps to remove oil coat and smaller molecules such as primers and dNTPs.

When PW-HGMs are loaded with amplification solution, the PW-HGMs are in effect compartmentize the amplification solution into uniform microdroplets of the same size of PW-HGMs. This microcompartmentization process is much simpler than forming an emulsion such as in bead emulsion PCR. In bead emulsion PCR, a successful clonal amplification reaction only occurs when a microdroplet contains a single bead and a single template molecule. This is usually attained at the sacrifice of efficiency. In order to make a water-in-oil emulsion stable enough under PCR cycling condition much high concentration additive such as detergent is used. Such condition is not optimal for PCR and loss of efficiency is unavoidable. In this invention, the use of PW-HGMs overcomes all these problems. The rigidity of PW-HGMs does not require an emulsion and the amplification of fragments is more uniform due to the uniformity of the microdroplets contained by the PW-HGMs. The condition of clonal amplification is also easier to control. Theoretically modeling indicates that if the ratio of template molecules to the number of PW-HGMs is set at 0.5, about 30% of PW-HGMs will have a single template molecule and 60% will not contain a template molecule and the rest 10% contain multiple template molecules. If this ratio increases to 1, about 37% of PW-HGMs will have a single copy template molecule and 37% contain no template and the rest have multiple template molecules. So the optimal ratio of template molecule to PW-HGMs is between 0.5 and 1. In bead emulsion PCR, adjustment of this bead to template ratio is complicated by a third variable—the formation of the liquid droplets.

Beside uniform amplification of different species of nucleic acid fragments in a complex fragment library, another major advantage of PW-HGMs as microreactors for clonal amplification of nucleic acid fragments is that the level of amplification can be easily controlled by size of the microdroplets confined by the PW-HGMs. This is not so easy with bead emulsion PCR technique. In emulsion PCR, when the droplets reach certain size the emulsion tend to destabilize and even collapse at a raised temperature. In contrast, PW-HGMs do not have such limitation. PW-HGMs as small as 1 micron and as big as 250 microns in diameter can be used in PCR. However, to generate emulsion droplets larger than 100 microns in diameter, a specialized device has to be employed (U.S. Pat. No. 7,851,184). Such specialized device, due to its limited throughput, introduce a bottleneck in the sample preparation procedure for high throughput sequencing.

The wall porosity of hallow glass microspheres is important but can only be controlled to certain level. This is usually done but controlling the time and temperature of the annealing process and the leaching time. The requirements for PW-HGMs in the present invention are quite different than those for hydrogen storage (U.S. Pat. No. 7,666,807), which require very fine and relatively uniform wall porosity. In this invention, we take advantage of the rigidity of the wall of the PW-HGMs to avoid the use of emulsion, the only requirement is that the porous wall must permit entry of large molecules such as polymerase and template nucleic acid molecules. Non-uniform porosity of the wall actually is an advantage because large molecules may enter the PW-HGMs through the large pores. It has been determine the pore size of PW-HGMs ranges from 2-900 nm (U.S. Pat. No. 7,666,807).

After loaded with amplification solution, the porous wall of PW-HGMs is easily sealed by a water immiscible phase and the PW-HGMs become isolated from one another even though they are in close contact with one and another. This guarantees that each PW-HGM serves as an independent microreactor in the PCR amplification process, thus avoiding cross contamination.

While PCR is the preferred method, other methods of nucleic acid amplification are well compatible with PW-HGMs. One useful amplification method that can take advantage of the PW-HGMs for clonal amplification is the rolling cycle nucleic acid amplification. The process of rolling cycle amplification is isothermal. The method requires a circular single stranded template and a primer that can hybridize to any region of the circular molecule (U.S. Pat. Nos. 6,183,960; 5,912,124; Fire and Xu, Proc. Natl. Acad. Sci. USA 92:4641-4645 (1995), Zhang, D Y, et al. Gene, 211(2): 277-285 (1998) and Lizardi, P M, et al., Nature Genetics, 19: 225-232 (1998).) In the presence of a polymerase with a strong displacement activity such as Phi 29 or Bst DNA polymerase and dNTPs and proper buffer environment, the polymerase will start DNA synthesis and proceed almost indefinitely, generating multiple copies of the circular template in a linear but concatenated form. Large DNA molecules (>50 kb) tend to be broken into smaller fragments in solution by shear force in normal liquid handling procedure such as pipetting. When this large concatamer is generated inside a PW-HGMs, it is equivalent to being locked inside a cage. Manipulate of such large molecule will become easy as they will be protected by the rigid wall of PW-HGMs and the molecule will not be susceptible to the shear force in solution. When the concatenated molecules are locked inside the PW-HGMs, the PW-HGMs can be placed in a solution containing dTNPs and other necessary components to further amplify the locked molecules. In such scenario, the PW-HGMs work like an artificial cell with the trapped DNA molecule replicating inside and the porous wall serving as an effective cell membrane for exchange of energy and necessary components to sustain the replication of DNA inside the PW-HGMs. An obvious application of such large multicopy concatenated nucleic acid molecules is for DNA sequencing. Such highly and clonally amplified fragment will immensely improve the detection sensitivity and thus increase the read length of current sequencing systems based on the principle of sequencing-by-synthesis.

The PW-HGMs assisted clonal fragment amplification method disclosed in this invention can be also applied to the detection of gene copy number in a genome or the number of transcripts in a transcriptom. This clonal amplification approach to copy number detection is similar to digital PCR (U.S. Pat. Nos. 7,915,015; 7,888,017). The gene targets to be detected are diluted to a level so that the ratio of targets to the PW-HGMs is less than 1. After PCR amplification, each PW-HGM will contain multiple copies of fragments from a single molecule and the copy number of a target gene or a sequence can be detected by counting the number of positive PW-HGMs carrying the amplified fragment. Counting positive PW-HGMs can be carried out using a flow cytometer when the DNA inside the PW-HGMs is stained with a DNA staining dye such as YOYO-3. Counting PW-HGMs can also be carried out using a fluorescent microscope or a microarray scanner.

### EXAMPLES

The examples provided herein are intended to serve as explanation of the present invention. It is obvious to those skilled in the art that various modifications can be made without deviating from the scope or spirit of the invention. Thus, the examples described are specific embodiments of the invention and they are not intended to limit any aspects of other possible embodiments that can be covered within the scope of the appended claims.

### Preparation of Porous Wall Hollow Glass Microspheres

The production of hollow glass microspheres has been a well known art (U.S. Pat. Nos. 4,661,137; 5,256,180, which are incorporated herein by reference). Hollow glass mcirospheres are now produced at an industrial scale as an inert and light filler material. Making porous structure in glass is also known in the art (U.S. Pat. Nos. 5,397,759, 5,225,123, and 7,666,807). This is usually involves treated the glass at a temperature that allow separation of silica rich phase from the borate rich phase. The borate rich phase can be removed by leaching with a strong acid such as HCl. The procedure described below was used to produce porous wall hollow glass microspheres which were used in the experiments described in the examples that follows.

Hollow glass microspheres of a mean diameter of 35 microns and density of about 0.3 g/cc were obtained from 3M. The hollow glass microspheres were heat treated at 630° C. in a muffle oven for 16-24 hours. Defective hollow glass microspheres were removed by flotation selection. The portion afloat in water was collected and treated in 3M HCl for 16 hours in a beaker at room temperature. The hollow glass microspheres with porous wall would sink to the bottom of the beaker. They were collected and rinsed extensively with distill water and kept therein until use.

### Loading PCR Amplification Reaction to PW-HGMs

PW-HGMs are better stored in pure water or proper buffer such as TE buffer to avoid absorption of impurities from the environments when stored in dry state. Also, PW-HGMs are much more convenient to aliquot into small quantities by pipetting than weighing. Two methods are equally effective for loading amplification reaction to PW-HGMs.

### Method A: Loading Amplification Reaction to Dry PW-HGMs.

PW-HGMs are first transferred to a microtube or a container of proper size and the excess solution can be removed by vacuum aspiration. The PW-HGMs are dried at 110-120° C. for 30-60 minutes to completely remove the water in the interior. Amplification solution is then mixed with the dry PW-HGMs. The mixture is incubated at 4° C. for 1-2 hours. Due to the capillary action of the pores on the wall, the amplification can fill in the interior void slowly. Completely filled PW-HGMs will settle to the bottom of the container. The filled PW-HGMs can be rinsed with reaction without template DNA and primers and then suspended in light mineral oil for PCR cycling.

### Method B: Loading Amplification Reaction to PW-HGMs Stored in Amplification Buffer.

The PW-HGMs are first equilibrated in a reaction buffer. The required amount of PW-HGMs is transferred together with the reaction buffer. Other necessary components such as polymerase, template DNA, primers, dNTPs, and additive such as BSA, betaine or Triton X-100 are added to a final proper concentration. And the mixture is stored at 4° C. for 6-12 hours to allow the components to diffuse to the interior of PW-HGMs. Since the porous wall is restrictive to the diffusion of large molecules an electric field may be applied to the mixture to actively move the macromolecules such as polymerase and template DNA through the porous wall. This is can be done by placing the mixture in a well of normal gel such as agarose gel and applying a electric field that changes directions at a proper frequency. Alternating the electric field will keep the components in the well but all the charged molecules will move back and for locally so as to facilitate the entry of macromolecules to the interior of PW-HGMs. Applying an electric field to load the amplification reaction to the PW-HGMs can be as short as a few minutes instead of hours by passive diffusion. It can be easily envisaged that one may develop a kit that offers such convenience.

In both method A and B, if PCR is to be used as the amplification method, proper measures must be taken to avoid primer-dimer formation. Formation of primer-dimer will result in lower efficiency in PCR and lead to failure in amplifying fragment from a single template molecule. A common way of limiting primer-dimer formation is by holding an essential component such as polymerase, dNTPs or Mg²⁺ until the PCR cycling starts. But for the format of amplification with PW-HGMs, a more convenient way is to load all the necessary components to the PW-HGMs but only trigger the polymerase activity at a raised temperature. There are two common ways to achieve this: 1) use a polymerase that is not active at room temperature but become active at 94° C. Such polymerases are called “Hot Start” polymerase which can be purchased from several vendors. 2) use a derivative of dNTPs that is not active at room temperature but is activated at a raised temperature. dNTPs having such property is now available commercially.

### Example 1

### Loading DNA Templates to PW-HGMs Under Two Conditions

This example shows that large DNA can enter the interior of PW-HGMs through diffusion or with the assistance of an electric field. PW-HGMs were incubated with a size ladder to check the permeability of the porous wall to fragments of different size. 5 μg of 50 bp DNA size ladder in 50 μl TE buffer was mixed with 100 μl of TE saturated PW-HGMs and incubated at room temperature for two hours. Half of the mixture is transferred to a well of agarose gel and an electric field of strength 3 volts/cm is applied. The electric field is reversed every 60 seconds. The sample is taken out of the well after 20 minutes of treatment and transferred to a well in a new gel. The untreated control sample together with size ladder was placed in adjacent wells and electrophorised for 50 minutes at 3.5 volts/cm to separate the ladder fragments. FIG. 2 shows results of PW-HGMs loaded under two different conditions. As seen, the porous wall does not seem to be a barrier to prevent DNA up to 1.3 kb from entering the PW-HGMs. Loading of DNA fragments can be greatly facilitated by an electric field.

### Example 2

### Using PW-HGMs as Microvessels for PCR

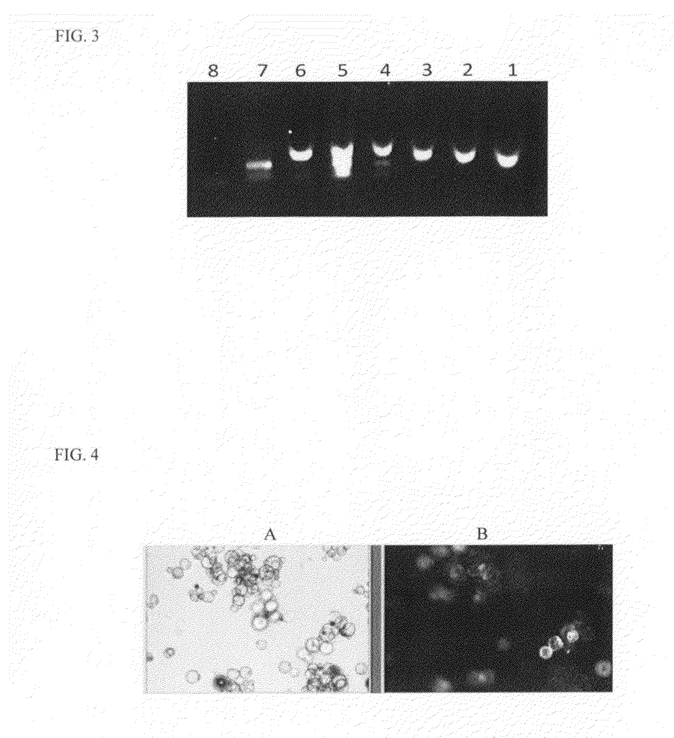

This example demonstrates that PW-HGMs can serve as effective microreactors for PCR. PW-HGMs were soaked in a PCR mix including Taq polymerase, buffer, template DNA, and primers at 4° C. 5 hrs to allow diffusion of template and Taq polymerase into the interior of the PW-HGMs. The template used was sheared mouse genomic DNA with an average size of about 600 bp. The primer sequences were: Fprimer1, TGAGCACATTGCTGTGACA and Rprimer1, CCAGGTCAGCGAGATGAAAT. The ratio of template molecules to the number of PW-HGMs was about 1. After incubation at 4° C., dNTPs were added to the mix and incubated on ice for 5-10 minutes. The PW-HGMs were transferred to cold PCR buffer to rinse off excess solution in the exterior of the PW-HGMs. The PW-HGMs were then suspended in light mineral oil and subjected to thermal cycling. The PCR products and their yield were analyzed by gel electrophoresis after the oil on the exterior wall of the PW-HGMs was cleaned off using a detergent solution composed of 50 mM Tris.-HCl, 100 mM KCl, and 0.1% Triton X-100. FIG. 3 shows the results of one experiment. We tested both normal Taq and truncated Taq polymerase and found that both showed little difference in amplification efficiency, indicating that the porous wall is not a barrier to prevent the passage of Taq polymerase.

### Example 3

### Assay to Detection Cross Contamination in PW-HGM Mediated PCR

We further tested the cross contamination of oil suspended PW-HGMs. PW-HGMs with template DNA were mixed with those without in mineral oil and performed PCR amplification to check if the oil layer on the exterior surface of PW-HGMs can prevent cross contamination between PW-HGMs. If there was cross contamination we would expect that the amplified products would spill to those PW-HGMs without template DNA and initiate amplification, resulting in all PW-HGMs carrying amplified products. After PCR the PW-HGMs were stained using DNA stain YOYO-3 (Invitrogen) at 0.1 μM and inspected under a fluorescence microscope. The ratio of empty PW-HGMs to PW-HGMs with amplified products was found to be the same as the initial ratio of empty PW-HGMs to template loaded PW-HGMs. This indicates that there is little or no cross contamination between PW-HGMs suspended in oil.

### Example 4

### Assay to Detection Cross Contamination in PW-HGM Mediated PCR

PCR products loaded PW-HGMs were soaked in TE buffer 2 hours at 4° C. without using a detergent solution to remove the oil layer on the PW-HGMs. The TE buffer was check for the presence of PCR product by normal PCR. After 30 cycles of PCR no amplification products were detected. In contrast, if the oil layer was removed PCR products were readily detectable on gel after 25 PCR cycles. This result further confirmed that the amplified products do not escape the oil coated porous wall to cause cross contamination.

### Example 5

### Retention of Amplified Fragments by PW-HGMs

A 250 bp genomic fragment was amplified in PW-HGMs by PCR. After removing the oil coat on the PW-HGMs using a rinse solution of composition of 50 mM Tris.-HCl, 100 mM KCl, and 0.1% Triton X-100, the PW-HGMs were dialyzed in TE buffer at 4° C. for 3 hours and then stained with 0.1 μM YOYO-3 and measured the average fluorescence of a population of PW-HGMs. The loss of average fluorescence as compared to that of PW-HGMs without dialysis was found in the range of 15-25%. This result suggests that diffusion of DNA fragments is sufficiently slow for convenient post amplification processing.

### Example 6

### Detection of Copy Number Using PW-HGM Based Clonal Amplification

This example demonstrates that clonal amplification of targets fragments in PW-HGMs can be used for detection of fragment copy number. Mouse female and male genomic DNA was sheared to about 600 bp by sonication. 0.1 μg of sheared genomic DNA was mixed with about 100,000 PW-HGMs in PCR buffer containing 0.2 mM dNTP, 0.1% Triton X-100, 5 units of KleanTaq (New England Biotech), a pair of primers targeting to chromosome X. The mixture was incubated at 4° C. overnight. Excess solution was removed by aspiration. The PW-HGMs were rinsed briefly with 50 mM Tris.-HCl, 100 mM KCl, and 0.1% Triton X-100, and immediately suspended in light mineral oil in a PCR tube which was subject to 30 PCR cycle of this program: 94° C. for 3 minutes to activate Taq polymerase and denature the template, PCR cycle: 94° C. 30 seconds, 55° C. 30 seconds, 72° C. 25 seconds, cycle number: 30. A final extension reaction at 72° C. for 2 minutes to finished the PCR amplification. PW-HGMs were cleaned off oil by rinsing in 50 mM Tris.-HCl, 100 mM KCl, and 0.1% Triton X-100 three times. PW-HGMs were stained with 0.1 μM YOYO-3 (Invitrogen) in TE with 20% mercaptoethanol for 5 minutes and spread out on a microscope slide for inspection under a fluorescent microscope coupled to a cool CCD camera. Bother phase contrast and fluorescence images of PW-HGMs were taken. The fluorescence positive PW-HGMs in the fluorescence image and the total PW-HGMs in the phase contrast image were counted. The average percentage of fluorescence positive PW-HGMs was calculated for bother male and female samples. In a typical experiment, 56-60% of PW-HGMs were found fluorescent in female DNA as compared to 27-30% of fluorescence positive PW-HGMs in the male DNA. This result clearly demonstrated that by scoring the clonally amplified PW-HGMs one can determine the copy number of a specific sequence in genomic DNA.

The advantages of the present invention include, without limitation, that the invention has overcome many limitations of the existing methods for parallel clonal amplification of a plurality of nucleic acids such as a genomic fragment library. The existing methods for parallel amplification involve very complicated procedures and require unreliable water-in-oil emulsion to encapsulate solid microbeads. The present invention has significantly simplified the amplification process.

In broad embodiment, the present invention is a novel method based on a novel concept of dividing liquid solution into very small but uniform compartments using porous wall hollow glass microspheres. The thin layer of oil coated on the microsphere surface serves as an insulator to prevent the internal content from diffusing out through the porous wall. And the individual PW-HGMs are essentially independent microreactors which can be mixed in a single tube for parallel manipulation such as PCR amplification.

While the foregoing description of the invention should enable one of ordinary skill to make and use what is considered presently to be the best mode thereof, those of ordinary skill will understand and appreciate the existence of variations, modifications, combinations, and equivalents of the specific embodiment, method, and examples herein. The invention should therefore not be limited by the above described embodiments, methods, and examples, but by all embodiments and methods within the scope and spirit of the invention as claimed.

### Sequence Listing

| | |
|---|---|
| Fprimer1, | TGAGCACATTGCTGTGACA |
| Rprimer1, | CCAGGTCAGCGAGATGAAAT |

What is claimed is:

1. A method of amplifying sequestered populations of nucleic acid molecules in parallel, comprising:
(a) Encapsulating one or more species of template nucleic acid molecules in a amplification solution into a plurality of porous wall hollow glass microspheres of 1 to 250 microns in diameter;
(b) Suspending said porous wall hollow glass microspheres in a water immiscible phase such as mineral oil;
(c) Amplifying the species of said template nucleic acid molecules sequestered within said porous wall hollow glass microspheres via polymerase chain reaction or isothermal multiple strand displacement reaction such as rolling cycle amplification in a amplification solution comprises a mixture of necessary components for performing an amplification reaction of the species of said template nucleic acid molecules.
(d) separating said porous wall hollow glass microspheres to collect the sequestered amplified products contained therein from the immiscible phase by washing said porous wall hollow glass microspheres with a detergent solution comprising 0.1 to 1% Triton X-100, thereby producing said porous wall hollow glass microspheres carrying amplified nucleic acid molecules.

2. The method of claim 1, wherein: the method of encapsulating said template nucleic acid molecules comprises filling the amplification solution to said porous wall hollow glass microspheres; mixing a solution of said template nucleic acid molecules with said porous wall hollow glass microspheres filled with said amplification solution; moving said template nucleic acid molecules into said porous wall hollow glass.

3. The method of claim 2, wherein: the method of filling said amplification solution to porous wall hollow glass microspheres comprises soaking dry said porous wall hollow glass microspheres in the amplification solution.

4. The method of claim 2, wherein: the method of moving said template nucleic acid molecules into said porous wall hollow glass microspheres is by incubating the mixture of aqueous solution and said porous wall glass microspheres at 4° C. to 10° C. from 1 to 5 hours.

5. The method of claim 2, wherein: the method of moving said template nucleic acid molecules into said porous wall hollow glass microspheres is by applying an electric field of a field strength 2-3 volts/cm to 10 volts/cm and an alternating frequency of 1 to 60 per minutes to the mixture of the aqueous amplification solution and said porous wall hollow glass microspheres.

6. A method for detection of copy number of a target nucleic acid sequence in a sample, comprising steps of:
(a) Forming a primer-initiated, template-dependent amplification solution containing said sample, primers targeting to said target nucleic acid sequence, and other necessary components supporting amplification such as polymerase, dNTPs, and so forth of said target nucleic acid sequence.
(b) Encapsulating said amplification solution into a plurality of porous wall hollow glass microspheres of 1 to 250 microns in diameter so that the ratio of the number of said porous wall hollow glass microspheres to the number of said target nucleic acid sequence is 1 to 10000.
(c) Suspending the amplification solution filled porous wall hollow glass microspheres in an oil phase and conducting polymerase chain or rolling cycle amplification reaction under proper conditions.
(d) Counting the number of porous wall hollow glass microspheres with amplification products.

7. The method of claim 6, wherein: the method of counting the number of porous wall hollow glass microspheres with amplification products comprising: Rinsing said porous wall hollow glass microspheres in TE buffer containing 0.1-0.5% Triton X-100; Staining said porous wall hollow glass microspheres in a solution containing a DNA intercalating dye such YOYO-3, cyber green, TOTO-3, and so forth; Suspending said porous wall hollow glass microspheres in a proper medium such as buffer solution, gel matrix, and so forth; Counting the fluorescent positive porous wall hollow glass microspheres via a flow cytometer, via a fluorescent microscope, or via microarray scanner.


SEQUENCE LISTING (2 sequences)

SEQ ID NO: 1 - DNA, 19 bp, Artificial
  Synthetic PCR primer
tgagcacattgctgtgaca

SEQ ID NO: 2 - DNA, 20 bp, Artificial
  Synthetic PCR primer
ccaggtcagcgagatgaaat